(19) United States
(12) Patent Application Publication
(10) Pub. No.: US 2011/0305740 A1
(43) Pub. Date: Dec. 15, 2011

(54) GRANULATED POWDER CONTAINING VEGETABLE PROTEINS AND MALTODEXTRINS, PROCESS FOR PRODUCING SAME, AND USES THEREOF

(75) Inventors: Bernard Boursier, Violaines (FR); Damien Passe, Douai (FR)

(73) Assignee: ROQUETTE FRERES, Lestrem (FR)

(21) Appl. No.: 13/203,035

(22) PCT Filed: Feb. 25, 2010
(86) PCT No.: PCT/FR2010/050328

§ 371 Date: Aug. 24, 2011

(30) Foreign Application Priority Data
Mar. 2, 2009 (FR) 0951294

(51) Int. Cl. A61K 8/02; A61K 38/02; A23K 1/00; A23L 1/052; A23G 9/38; A61K 9/14; A61Q 90/00

(52) U.S. Cl. 424/401; 424/489; 514/5.5; 426/656; 426/578; 426/565

(57) ABSTRACT

The present invention concerns a granulated powder comprising at least one vegetable protein and at least one starch hydrolyzate, characterized in that it has a laser volume average diameter D4,3 of between 10 μm and 500 μm, preferably between 50 μm and 350 μm, and even more preferably between 70 μm and 250 μm, and a dry matter content, determined after stoving at 130° C. for 2 hours, of greater than 80%, preferably greater than 85%, and even more preferably greater than 90%.

The present invention also concerns process for manufacturing this granulated powder its use in various industrial field, and more particularly in the food-processing field, where it is used as a functional agent such as an emulsifying, overrun, stabilizing, thickening and/or gelling agent, in particular for totally or partially replacing certain animal proteins in the preparation of food products.

### FIELD OF THE INVENTION

The subject of the present invention is a granulated powder containing vegetable proteins and maltodextrins and/or glucose syrups, and also the process for producing same and the uses thereof.

### TECHNICAL BACKGROUND

Dietary habits have altered profoundly in industrialized countries since the Second World War and even more recently driven by the food-processing industry, the increasing influence of which on the nutritional behavior of populations tends to gradually blur the differences related to the conventional nutritional habits. This change probably contributes to increasing the risks of lithiasis, cardiovascular risks, and the risks of diabetes, obesity and certain cancers of nutritional origin in industrial societies where the daily energy needs have a tendency to become reduced in an increasing number of individuals with increasingly sedentary activity.

Proteins represent, after carbohydrates and lipids, the third major energy source in our diet. They are provided both by products of animal origin (meats, fish, eggs, dairy products) and by plant foods (cereals, legumes, etc.). Daily protein needs are between 12% and 20% of the food intake. In industrialized countries, these intakes are predominantly in the form of proteins of animal origin. Studies show that we are consuming too many proteins of animal origin (70% of our intakes on average) and not enough vegetable proteins (30%). In addition, our food is too high in lipids, in particular in saturated fatty acids, and in sugars, and too low in fibers. In terms of protein intake, insufficiency like excess is prejudicial: in the event of insufficient intake, there is a risk of development and growth being disturbed. In the event of excessive intake, the amino acids constituting the proteins are oxidized or converted to carbohydrates or to fats. Such an excess is perhaps not without unfavorable consequences, especially in the case of animal proteins: in addition to the actual risk of oxidation and conversion of amino acids, it should be remembered that foods high in animal proteins are often also high in lipids and in saturated fatty acids. A recent study implicates the responsibility of excess animal proteins in the generation of subsequent obesity.

In addition, the advantages for the health are obvious since excessive consumption of animal proteins has been brought to the fore in causes for the increase in certain cancers and cardiovascular diseases.

In addition, intensive farming of animals generates serious environmental problems. Meat production requires twice as much water and two to four times more space than the production necessary for a plant-based diet. Animal farming also represents considerable soil and air pollution. It was recently proved that pollution from cattle farming exceeded motor vehicle pollution in terms of nitrogen waste.

Finally, animal farming represents a formidable waste of the world's water resources: 7 kg of cereals are necessary to produce 1 kg of beef-4 kg for producing 1 kg of pork-2 kg for producing 1 kg of poultry. Farm animals are fed with cereals that are edible for humans, such as soybean (the term then used is cake) and corn. In Brazil, soybean is today the main cause of deforestation of the Amazonia.

Thus, animal proteins derived from meat have many disadvantages, both in terms of health and in terms of environment.

In parallel, animal proteins derived from milk or from eggs can be allergenic, leading to reactions which are very bothersome, or even dangerous, in everyday life.

Thus, eggs are food allergens (a type of allergen) which penetrate via the digestive tract and which, in certain individuals, can cause a release of histamine by the cells of the organism. It is this substance which is responsible for the symptoms of inflammation and which leads to contraction of the bronchial muscles. Hypersensitivity is most commonly related to the egg white. On the other hand, in some individuals it is the proteins contained in the yolk which cause allergic reactions. Egg allergy is particular since it causes the entire range of symptoms associated with food allergies, such as bloating, digestive problems, skin rashes, nausea, diarrhea, asthma attacks and eczema. Egg white allergy can go as far as anaphylactic shock, a violent reaction which can lead to the death of the allergic individual if the latter does not immediately receive an injection of adrenalin.

Dairy product allergy is one of the most widespread allergic reactions. Studies demonstrate that 65% of individuals who suffer from food allergies are allergic to milk. The adult form of milk allergy, herein referred to as “dairy products allergy”, is a reaction of the immune system which creates antibodies in order to combat the unwanted food. This allergy is different than cow's milk protein (bovine protein) allergy, which affects newborns and infants. Dairy product allergy causes varied symptoms, such as constipation, diarrhea, flatulence, eczema, urticaria, nausea, migraines, infections, abdominal cramps, nasal congestion and even serious asthma attacks. Allergic individuals should completely eliminate milk, dairy products and derivatives thereof from their diet. The following terms are indicators of the presence of cow's milk or derivatives thereof in the ingredients of a product: buttermilk, calcium caseinate, sodium caseinate, casein, caseinate, hydrolyzed casein, dried milk solids, lactalbumin, lactose, lactoglobulin, low-fat milk, milk powder, condensed milk and whey.

Another major problem associated with milk proteins is their cost, which never ceases to increase. The application of milk quotas has caused, on the one hand, a drastic reduction in the amount of milk proteins available for the production of food products and, on the other hand, large fluctuations in their price. Manufacturers are increasingly seeking substitute products for these milk proteins.

In view of all the disadvantages, whether they are economical, environmental or nutritional, associated with the consumption of animal proteins derived from meat and/or derived products, there is, as a result, great interest in the use of substitute proteins, also called alternative proteins, classified among which are vegetable proteins. The alternative market for these proteins is developing rapidly, for many reasons. These proteins have a profound influence on the formulation of balanced foods and diets based on a low glycemic index (GI) and a high protein intake, and conventional manufacturers are beginning to seek new sources of proteins in order to enrich their products.

For example, document WO 2008/066308 describes a food composition containing an optimum combination of nutrients essential for a balanced diet, combined with soybean proteins. This composition makes it possible to reduce the problems of obesity by reducing, inter alia, harmful protein intakes.

Document EP 0522800 describes a novel method for treating a vegetable protein concentrate to enhance its functionality for binding fat and water and also its use as a replacement for animal proteins in the manufacture of sausages.

Document EP 0238946 describes an improved protein isolate derived from seeds of a grain legume with a relatively low lipid content, the method for preparing same and also the use thereof as an additive in the manufacture of sausages and saveloys.

The applicant company also focused in on this research in order to be able to meet the increasing demands from manufacturers for compounds having advantageous functional properties without however, having the drawbacks of certain already-existing compounds.

Specifically in fields as diversified as nutrition, pharmacy, cosmetics, agrochemistry, construction materials and paper-cardboards, manufacturers are constantly searching for new compounds which have a positive and beneficial image in terms of health and which are capable of modifying the functional properties of media in order to manufacture products having varied textures.

Thus, the applicant has carried out considerable research studies on Vegetable Protein Materials (VPM) as food ingredients. This interest in VPM is first of all due to their numerous functional properties, but also to advantageous nutritional qualities by virtue of their “essential” amino acid composition.

In the present application, the term “VPM” denotes food ingredients obtained from oleaginous plants, leguminous plants or cereals by reduction or elimination of some of the main non-protein constituents (water, oil, starch, other carbohydrates), so as to obtain a protein content (N×6.25) of 50% or more. The protein content is calculated on the basis of the dry weight excluding the vitamins and mineral salts.

VPM are increasingly used in food applications. They have become an important ingredient owing to their overrun, texturing, emulsifying, thickening, stabilizing, foaming or gelling properties, which are constantly being improved, for use in known applications or else quite simply in completely new creations.

One of the objects of the present invention is therefore to propose vegetable proteins as a replacement for animal proteins, while at the same time making it possible to retain, in a product in which they are used, functional properties, a flavor and palatability and also a nutritional value which are at least similar, or even improved. The product will have an equivalent nutritional value:
if its protein quality is not inferior to that of the product of origin, and
if it contains an amount of proteins (N×6.25), mineral salts and vitamins equivalent to that present in the products of animal origin.

Proteins play a major role in the organoleptic quality of many fresh or manufactured foods, for instance the consistency and the texture of meat and meat products, of milk and derivatives, of pasta and of bread. These food qualities very frequently depend on the structure and the physicochemical properties of the protein components or quite simply of their functional properties.

In the present application, the term “functional properties of food ingredients” means any non-nutritional property which influences the usefulness of an ingredient in a food. These various properties would contribute to obtaining the desired final characteristics of the food. Some of these functional properties are solubility, hydration, viscosity, coagulation, stabilization, texturing, dough formation, and foaming and coagulating properties.

In addition to the substitution of animal proteins and, as a result, the elimination of many of the disadvantages associated with their use, the applicant company has also concentrated on the formation of novel ready-to-use food ingredients, containing, in addition to the VPM, other compounds having different but complementary functional and/or nutritional properties.

Indeed, nowadays, in the interests of maximum cost-effectiveness, there is an increasing desire to simplify manufacturing processes on the part of manufacturers, and most particularly in the food-processing industry.

This simplifying of food product manufacturing processes results in particular in a reduction in the number of compounds used, and in particular in the ingredients involved in preparing the final products. This reduction in ingredients makes it possible both to limit the manufacturing times of the products, to simplify the manufacturing processes and to reduce the costs thereof. However, it must not alter the texture or any of the functional, nutritional, sensory or organoleptic properties of said products.

Still with a desire to simplify food product manufacturing processes, manufacturers are also increasingly demanding with respect to the form of said ingredients used. The dry form is by far the form preferred by manufacturers, whether in terms of preservation, storage or handling, compared with a liquid form for example, which is much less stable over time. Nevertheless, the use of ingredients in pulverulent form has the disadvantage that these products are sometimes difficult to dissolve, which can lead to settling out, and poor dispersibility with the formation of lumps and therefore uneven distribution of the ingredients during the process. What is more, the handling of pulverulent products poses safety problems due, inter alia, to the dry residues that handlers may breathe in, with, in addition, risks of fire and explosion.

As a result of all the above, there is a real, unmet need to have a composition used as a substitute for proteins of animal origin, which has several advantageous functional properties enabling it to reduce the number of additives used in the manufacture of a finished product while at the same time providing it with technological characteristics similar to those obtained by using said additives separately, and which is in a dry but nonpulverulent form which can be easily hydrated.

Armed with this observation and after a considerable amount of research, the applicant company has, to its credit, reconciled all these objectives reputed up until now to be difficult to reconcile, by proposing a novel composition containing, inter alia, vegetable proteins, characterized in that it:
combines a vegetable protein and a starch hydrolyzate, itself having an advantageous and desired functional characteristic and/or nutritional characteristic and/or technological characteristic,
is in dry but nonpulverulent form, i.e. in granular form, it is referred to as a granulated powder,
has a dry matter content of greater than 80%, preferably greater than 85%, and even more preferably greater than 90%,
has an “instant” nature, i.e. this granulated powder has very good wettability, dispersibility and solubility in water.

Said granulated powder is characterized in that it exhibits, compared with the simple physical mixtures of powder described in the prior art, better dispersion in water and better dissolution under cold conditions, and better flowability for metering operations, and in that it offers a better environment for handling the powders owing to the absence of dust. What is more, this granulated powder has improved functional characteristics, that the simple physical mixing of the various constituents would not have made it possible to obtain.

### SUMMARY OF THE INVENTION

The subject of the present invention is therefore a granulated powder comprising at least one protein of vegetable origin and at least one starch hydrolyzate, characterized in that it has a laser volume mean diameter D4,3 of between 10 μm and 500 μm, preferably between 50 μm and 350 μm, and even more preferably between 70 μm and 250 μm, and a dry matter content, determined after stoving at 130° C. for 2 hours, of greater than 80%, preferably greater than 85%, and even more preferably greater than 90%.

The present invention also relates to the process for producing this granulated powder and to the use thereof in various industrial fields, and more particularly in the food-processing field, where it is used as a functional agent such as an emulsifier, overrun, stabilizing, thickening and/or gelling agent, in particular for totally or partially replacing certain animal proteins in the preparation of food products.

### DETAILED DESCRIPTION OF EMBODIMENTS

The present invention relates to a granulated powder comprising at least one vegetable protein and at least one starch hydrolyzate, characterized in that it has a laser volume mean diameter D4,3 of between 10 μm and 500 μm, preferably between 50 μm and 350 μm, and even more preferably between 70 μm and 250 μm, a dry matter content, determined after stoving at 130° C. for 2 hours, of greater than 80%, preferably greater than 85%, and even more preferably greater than 90%.

In the present invention, said granulated powder is characterized in that the weight ratio of the vegetable protein to the starch hydrolyzate is between 99:1 and 1:99, preferably between 80:20 and 20:80, even more preferably between 65:35 and 35:65, and in particular between 55:45 and 45:55.

In the present invention, said granulated powder is characterized in that the sum of the amounts of vegetable protein and of starch hydrolyzate is between 30% and 100%, and preferably between 50% and 100%, of the total mass of said granulated powder (dry/dry).

In the present invention, the term “vegetable protein” denotes all proteins derived from cereals, oleaginous plants, leguminous plants and tuberous plants.

In the present invention, the term “vegetable protein” also denotes all proteins derived from algae and from microalgae.

These vegetable proteins can be used alone or as mixtures, chosen from the same family or from different families.

Thus, said granulated powder according to the invention is characterized in that the vegetable protein is a protein derived from the family of cereals, oleaginous plants, leguminous plants, tuberous plants, algae and microalgae, used alone or as a mixture, chosen from the same family or from different families.

In the present invention, the terms “algae” and “microalgae” are intended to mean eukaryotic organisms devoid of roots, stalks and leaves, but having chlorophyll and also other pigments that are incidental to oxygen-producing photosynthesis. They are blue, red, yellow, golden and brown, or else green. They represent more than 90% of marine plants and 18% of the vegetable kingdom, and comprise 40 000 to 45 000 species. Algae are organisms that are extremely varied both in terms of their size and their shape and in terms of their cell structure. They live in an aquatic or very humid environment. They contain many vitamins and trace elements, and are true concentrates of active agents that are stimulants of and beneficial to health and beauty. They have anti-inflammatory, hydrating, soothing, regenerating, firming and anti-aging properties. They also have “technological” characteristics which make it possible to give a food product texture. Specifically, the much-vaunted additives E400 to E407 are in fact merely compounds extracted from algae, the thickening, gelling, emulsifying and stabilizing properties of which are used.

Microalgae in the strict sense are undifferentiated unicellular or multicellular microscopic algae; they are photosynthetic microorganisms separated into two polyphyletic groups: eukaryotes and prokaryotes. They live in strongly aqueous environments, and can have flagellar mobility.

According to one preferred embodiment, the microalgae are chosen from the group consisting of Chlorella, Spirulina and Odontella.

According to an even more preferred embodiment, the microalgae of the present invention are derived from the Chlorella genus, and preferably from Chlorella vulgaris, Chlorella pyrenoidosa, Chlorella regularis or Chlorella sorokiniana, and even more preferably from Chlorella vulgaris.

In the present application, the term “cereals” is intended to mean cultivated plants of the grass family producing edible seeds, for instance wheat, oats, rye, barley, corn, sunflower, sorghum or rice. The cereals are often milled in the form of flour, but are also as grains and sometimes in whole-plant form (fodders).

In the present application, the term “tuberous plants” is intended to mean all the storage organs, which are generally underground, which ensure plant survival during the winter season and often plant multiplication by the vegetative process. These organs are bulging owing to the accumulation of storage substances. The organs transformed into tubers may be:
the root: carrot, parsnip, cassava, konjac,
the rhizome: potato, Jerusalem artichoke, Japanese artichoke, sweet potato,
the base of the stalk (more specifically the hypocotyl): kohlrabi, celeriac,
the root+hypocotyl combination: beetroot, radish.

In the present application, the term “oleaginous plants” denotes plants cultivated specifically for their seeds or their fruits rich in fats, from which oil for dietary, energy or industrial use is extracted, for instance rapeseed, groundnut, sunflower, soybean, sesame and the castor oil plant.

For the purpose of the present invention, the term “leguminous plants” is intended to mean any plants belonging to the family Caesalpiniaceae, the family Mimosaceae or the family Papilionaceae, and in particular any plants belonging to the family Papilionaceae, for instance pea, bean, broad bean, horse bean, lentil, alfalfa, clover or lupine.

This definition includes in particular all the plants described in any one of the tables contained in the article by R. Hoover et al., 1991 (Hoover R. (1991) “Composition, structure, functionality and chemical modification of legume starches: a review” Can. J. Physiol. Pharmacol., 69 pp. 79-92).

According to one preferred embodiment of the present invention, the vegetable protein belongs to the leguminous plants proteins.

According to another preferred embodiment, the leguminous plants protein is chosen from the group comprising pea, bean, broad bean and horse bean, and mixtures thereof. According to another preferred embodiment, the leguminous plants protein is chosen from the group comprising alfalfa, clover, lupine, pea, bean, broad bean, horse bean and lentil, and mixtures thereof, and preferably from pea, bean, broad bean and horse bean, and mixtures thereof.

Even more preferably, said leguminous plant protein is pea.

The term “pea” is here considered in its broadest sense, and includes in particular:
all wild-type varieties of smooth pea and of wrinkled pea, and
all mutant varieties of smooth pea and of wrinkled pea, irrespective of the uses for which said varieties are generally intended (food for human consumption, animal feed and/or other uses).

Said mutant varieties are in particular those known as “r mutants”, “rb mutants”, “rug 3 mutants”, “rug 4 mutants”, “rug 5 mutants” and “lam mutants” as described in the article by C-L Heydley et al., entitled “Developing novel pea starches” Proceedings of the Symposium of the Industrial Biochemistry and Biotechnology Group of the Biochemical Society, 1996, pp. 77-87.

Even more preferably, said leguminous plant protein is smooth pea.

Indeed, pea is the leguminous plant with protein-rich seeds which, since the 1970s, has been most widely developed in Europe and mainly in France, not only as a protein source for animal feed, but also for human diet.

The pea proteins are, like all leguminous plants proteins, made up of three main classes of proteins: globulins, albumins and “insoluble” proteins.

The value of pea proteins lies in their good emulsifying capacities, their lack of allergenicity and their low cost, which makes an economical functional ingredient.

Furthermore, the pea proteins contribute favorably to sustainable development and their carbon impact is very positive. This is because the pea cultivation is environmentally friendly and does not require nitrogenous fertilizers, since pea fixes nitrogen from the air.

Besides, in native globular form, pea proteins are water-soluble, which makes it possible to envision incorporating them into emulsions.

According to the present invention, the term “pea protein” preferably denotes the pea proteins which are mainly in native globular form, globulins, or albumins.

Even more preferably, the vegetable proteins, and in particular the pea proteins, used according to the invention are in the form of a composition of vegetable protein, and in particular pea protein, having:
a total protein content (N×6.25), expressed in grams of dry product, of at least 60% by weight of dry product. Preferably, in the context of the present invention, use is made of a protein composition having a high protein content of between 70% and 97% by weight of dry product, preferably between 76% and 95%, even more preferably between 78% and 88%, and in particular between 78% and 85%,
a soluble protein content, expressed according to a test for measuring the water-solubility of proteins, of between 20% and 99%. Preferably, in the context of the present invention, use is made of a protein composition having a high soluble protein content of between 35% and 95%, preferably between 45% and 90%, even more preferably between 50% and 80%, and in particular between 55% and 75%.

In order to measure the total protein content, the soluble nitrogenous fraction contained in the sample can be quantitatively determined according to the Kjeldahl method, and then the total protein content is obtained by multiplying the nitrogen content, expressed as percentage weight of dry product, by the factor 6.25. This method is well known to those skilled in the art.

In the present invention, the total protein content can also be measured by quantitatively determining the soluble nitrogenous fraction contained in the sample according to the method of A. Dumas, 1831, Annales de chimie [Annals of chemistry], 33, 342, as cited by Buckee, 1994, in Journal of the Institute of Brewing, 100, pp. 57-64, and then the total protein content is obtained by multiplying the nitrogen content, expressed as percentage weight of dry product, by the factor 6.25. This method, also known as the combustion method for determining nitrogen, consists of total combustion of the organic matrix under oxygen. The gases produced are reduced by copper and then dried, and the carbon dioxide is trapped. The nitrogen is then quantified using a universal detector. This method is well known to those skilled in the art.

To determine the soluble protein content, the content of proteins soluble in water of which the pH is adjusted to 7.5+/−0.1 using a solution of HCl or NaOH is measured by means of a method of dispersion of a test specimen of the sample in distilled water, centrifugation and analysis of the supernatant. 200.0 g of distilled water at 20° C.+/−2° C. are placed in a 400 ml beaker, and the whole is stirred magnetically (magnetic bar and rotation at 200 rpm). Exactly 5 g of the sample to be analyzed are added. The mixture is stirred for 30 min, and centrifuged for 15 min at 4000 rpm. The method for determining nitrogen is carried out on the supernatant according to the method previously described.

These vegetable protein, and in particular pea protein, compositions preferably contain more than 50%, 60%, 70%, 80% or 90% of proteins of more than 1000 Da. In addition, these vegetable protein, and in particular pea protein, compositions preferably have a molecular weight distribution profile consisting of:
1% to 8%, preferably from 1.5% to 4%, and even more preferably from 1.5% to 3% of proteins of more than 100 000 Da,
20% to 55%, preferably from 25% to 55% of proteins of more than 15 000 and of at most 100 000 Da,
15% to 30% of proteins of more than 5000 and of at most 15 000 Da,
and from 25% to 55%, preferably from 25% to 50%, and even more preferably from 25% to 45% of proteins of at most 5000 Da.

The determination of the molecular weights of the proteins constituting said vegetable protein, and in particular pea protein, compositions is carried out by size exclusion chromatography under denaturing conditions (SDS+2-mercaptoethanol); the separation is carried out according to the size of the molecules to be separated, the molecules of large size being eluted first.

Examples of pea protein compositions according to the invention, and also the details of the method for determining the molecular weights, can be found in patent WO 2007/017572, of which the applicant company is also the proprietor.

According to the present invention, said vegetable proteins, and in particular pea proteins, used for producing the granulated powder can also be “vegetable protein concentrates” or “vegetable protein isolates”, preferably “pea protein concentrates” or “pea protein isolates”. The vegetable protein, and in particular pea protein, concentrates and isolates are defined from the viewpoint of their protein content (cf. the review by J. Gueguen from 1983 in Proceedings of European congress on plant proteins for human food (3-4) pp 267-304)
the vegetable protein, and in particular pea protein, concentrates are described as having a total protein content of from 60% to 75% with respect to dry matter, and
the vegetable protein, and in particular pea protein, isolates are described as having a total protein content of 90% to 95% with respect to dry matter,
   the protein contents being measured by the Kjeldahl method (cf. above), the nitrogen content being multiplied by the factor 6.25.

In another embodiment of the present invention, the vegetable protein, and in particular pea protein, compositions that can be used may be “vegetable protein hydrolyzates”, preferably “pea protein hydrolyzates”. The vegetable protein, and in particular pea protein, hydrolyzates are defined as preparations obtained by enzyme hydrolysis or chemical hydrolysis, or by both simultaneously or successively, of vegetable proteins, and in particular pea proteins. The protein hydrolyzates are composed of a mixture of peptides of various sizes and of free amino acids. This hydrolysis can have an impact on the solubility of the proteins.

The enzyme and/or chemical hydrolysis is, for example, described in patent application WO 2008/001183. Preferably, the protein hydrolysis is not complete, i.e. does not result in a composition comprising only or essentially amino acids and small peptides (from 2 to 4 amino acids). Thus, the hydrolyzates according to the invention are not HPV compositions. The preferred hydrolyzates comprise more than 50%, 60%, 70%, 80% or 90% of proteins of more than 500 Da.

The processes for preparing protein hydrolyzates are well known to those skilled in the art and can, for example, comprise the following steps: dispersion of the proteins in water so as to obtain a suspension, hydrolysis of this suspension by means of the chosen treatment. Most commonly, it will be an enzymatic treatment combining a mixture of various proteases, optionally followed by a thermal treatment intended to inactivate the enzymes that are still active. The solution obtained can then be filtered through one or more membranes so as to separate the insoluble compounds, optionally the residual enzyme, and the high-molecular-weight peptides (greater than 10 000 daltons).

According to the present invention, the granulated powder comprises at least one vegetable protein and at least one starch hydrolyzate.

In the present invention, the term “starch hydrolyzate” denotes any product obtained by acid or enzymatic hydrolysis of legume, cereal or tuber starch. Various processes of hydrolysis are known and have been described, in general, on pages 511 and 512 of the Kirk-Othmer Encyclopedia of Chemical Technology, 3^{rd} Edition, Vol. 22, 1978. These hydrolysis products are also defined as purified and concentrated mixtures formed from linear chains made up of D-glucose units and of D-glucose polymers which are essentially α(1→4)-linked, with only 4% to 5% of α(1→6) branched glucosidic linkages, that have extremely varied molecular weights and are completely soluble in water. Starch hydrolyzates are very well known and completely described in the Kirk-Othmer Encyclopedia of Chemical Technology, 3^{rd} Edition, Vol. 22, 1978, pp. 499 to 521.

Thus, in the present invention, the starch hydrolysis product is chosen from maltodextrins, glucose syrups, dextrose (crystalline form of α-D-glucose) and any mixtures thereof.

The distinction between the starch hydrolysis products is based mainly on the measurement of their reducing power, conventionally expressed by the notion of dextrose equivalent or DE. The DE corresponds to the amount of reducing sugars, expressed as dextrose equivalent per 100 g of dry matter of the product. The DE therefore measures the strength of the starch hydrolysis, since the more the product is hydrolyzed, the more small molecules (such as dextrose and maltose, for example) it contains and the higher its DE is. Conversely, the more large molecules (polysaccharides) the product contains, the lower its DE is.

From the regulatory point of view, and also for the purpose of the present invention, the maltodextrins have a DE of from 1 to 20, and the glucose syrups have a DE of greater than 20.

Such products are, for example, the maltodextrins and the dehydrated glucose syrups sold by the applicant under the name Glucidex® (available DE=1, 2, 6, 9, 12, 17, 19 for the maltodextrins, and DE=21, 29, 33, 38, 39, 40, 47 for the glucose syrups). Mention may also be made of the glucose syrup sold by the applicant under the name “Roquette sirops de glucose”.

According to one advantageous embodiment of the present invention, the granulated powder comprises at least one vegetable protein and at least one maltodextrin.

According to a first variant of the invention, the granulated powder comprises a pea protein and at least one maltodextrin.

According to one advantageous embodiment of this first variant, the granulated powder comprises a pea protein and a maltodextrin having a DE of between 15 and 19.

According to a second variant of the invention, the granulated powder comprises a pea protein associated with a glucose syrup, the DE of which does not exceed the value of 47, and preferably 35.

According to a third variant of the invention, the granulated powder comprises a pea protein and a mixture of maltodextrins and of glucose syrup.

According to one advantageous embodiment of this third variant, the granulated powder comprises a pea protein and a mixture of maltodextrin having a DE of between 15 and 19 and of glucose syrup, the DE of which does not exceed the value of 47, and preferably 35.

In the context of the present invention, the expression “granulated powder” signifies that there is intimate mixing between the various components of this powder, that their distribution within the powder is substantially homogeneous, and that they are not only linked to one another by simple physical mixing. Interactions between the constituents can occur both outside the particle and inside.

In one particular embodiment, the granulated powder is not coated.

Conversely, in the present invention, the expression “simple mixing” signifies that there is no intimate mixing between the various constituents, and that there has only been simple physical mixing by contact. There is no interaction between the constituents since they are virtually not in contact with one another.

Indeed, in order to produce said granulated powder, the applicant company has noted that it is advisable to use a mixture of at least one vegetable protein and at least one starch hydrolyzate, and to modify its physical characteristics by employing a suitable process, such that very advantageous functional properties, which cannot be obtained if each compound is used separately or if the compounds are used simultaneously but in the form of a simple mixture of powders, are simultaneously obtained.

In the present invention, said granulated powder is prepared by means of a drying process according to a technique chosen from the group consisting of spray-drying, granulation or extrusion or of any other drying means known to those skilled in the art, and under conditions suitable for the chosen equipment, capable of enabling the production of a granulated powder according to the invention.

Thus, the present invention is also directed toward a process for manufacturing the abovementioned granulated powder. Said manufacturing process consists in drying conjointly at least two constituents, and comprises a step of bringing at least one vegetable protein into intimate contact with at least one starch hydrolyzate, it being possible for this step of bringing into intimate contact to be carried out according to any process known to those skilled in the art, and in particular according to a technique chosen from spray-drying, granulation and extrusion, and any combination of at least two of these techniques, such that said step of bringing into intimate contact results in a dry matter content, determined after stoving at 130° C. for 2 hours, of greater than 80%, preferably greater than 85%, and even more preferably greater than 90%. By way of example, mention will be made of a process for manufacturing said granulated powder according to a single spray-drying technique, or according to a single granulation technique, or else according to a combination of a spray-drying technique followed by a granulation technique.

Thus, according to a first variant of the invention, said granulated powder can be produced according to a manufacturing process which comprises a step of spray-drying of a suspension of at least one vegetable protein and of at least one starch hydrolyzate, said spray-drying step being followed by a step of granulation of the “spray-dried” powder on a granulator. According to this first variant, a suspension to be spray-dried is prepared, containing at least one protein of vegetable origin, preferably a pea protein, and at least one starch hydrolyzate, and preferably a maltodextrin having a DE of between 15 and 19, in the required proportions. Still according to this variant, it is also possible to envision preparing one aqueous suspension to be spray-dried per constituent.

Still according to this variant, the suspension to be spray-dried can be prepared either from a dry composition of vegetable proteins, and preferably from a dry composition of pea proteins, i.e. in the form of a powder which is then diluted in water, or from a floc of vegetable proteins, and preferably of pea proteins.

In this second alternative, the floc of vegetable proteins, and preferably the floc of pea proteins, is obtained by milling the vegetable flour, and preferably the pea flour, resuspending this milled flour in water, and then fractionating said suspension by any means known, moreover, to those skilled in the art, so as to isolate a protein-rich fraction. The proteins are then isolated from this fraction by means of a technique chosen from the group of techniques for precipitating proteins at their isoelectric pH and ultrafiltration-type membrane separation techniques. Finally, the separation of the precipitate (also referred to as “floc”) containing the soluble proteins is carried out on a centrifugal decanter or on a plate separator. The floc can be used as it is or suspended, depending on its dry matter content.

The spray-drying step is a unit drying operation which consists in converting into a powder a liquid, sprayed in the form of droplets brought into contact with a hot gas. This operation determines the size of the droplets produced (and their size grading), their path, their speed and, consequently, the final dimension of the dry particles, as well as the properties of the powders produced: flow, instant nature related to their solubility, density, compressibility, friability, etc.

The spray-drying step can be carried out in a spray dryer or a spray-drying tower, in which said suspension (or the suspensions) to be dried is (or are) divided in a stream of hot gas which provides the heat necessary for evaporating the solvent and absorbs, in order to evacuate it, the moisture released by the product during drying. The liquid mixture is introduced at the top via a nozzle or a turbine, and the “spray-dried” powder produced is harvested at the bottom of the tower. The dry solid is separated from the spray-drying gas by means of a cyclone (or cyclones), or by filtration (sleeve filter, for example). In certain cases, if this is found to be necessary, the tower can be filled with an inert gas in order to prevent oxidation phenomena.

The granulation step is carried out after the spray-drying step, and consists in spraying an aqueous solution onto the powder resulting from the spray-drying step. Such an operation, combining a spray-drying step followed by a granulation step, is conventionally carried out in a multi-effect spray dryer such as, for example, an MSD (multi-stage dryer) tower.

According to one preferred embodiment of this first variant, the process can be carried out according to the following steps:
1) preparing, at a temperature between 15 and 70° C., and preferably between 15 and 50° C., a suspension of pea proteins and of starch hydrolyzates, in which:
   said pea proteins have a soluble protein content of between 20% and 99%, preferably between 45% and 90%, even more preferably between 50% and 80%, and in particular between 55% and 75%;
   said starch hydrolyzates are chosen from the group consisting of maltodextrins of which the DE is between 15 and 19 and glucose syrups of which the DE does not exceed 47, and preferably 35, and any mixtures thereof;
   the weight ratio of the pea proteins to the starch hydrolyzates is between 99:1 and 1:99, preferably between 80:20 and 20:80, even more preferably between 65:35 and 35:65, and in particular between 55:45 and 45:55;
   the dry matter content of the suspension is between 25% and 50%, preferably between 30% and 40%,
1′) carrying out an optional first step of heat treatment at high temperature and for a short period of time in order to reduce the bacteriological risks of the suspension obtained according to 1, it being possible for said treatment to be chosen from HTST (high temperature short time) and UHT treatments;
1″) carrying out an optional second step of high-pressure homogenization of the suspension obtained according to 1), and independently of the optional first step;
2) maintaining said suspension of pea proteins and of starch hydrolyzates at a temperature of between 15 and 80° C., and preferably between 15 and 50° C., or, in the event of step 1′) being carried out, bringing said suspension of pea proteins and of starch hydrolyzates back to a temperature of between 15 and 80° C., and preferably between 15 and 50° C.;
3) spray-drying said suspension in an MSD-type spray-drying tower equipped with a high-pressure spray-drying nozzle with recycling of the fine particles at the top of the tower;
4) granulating in said spray-drying tower;
5) recovering the resulting granulated powder and comprising the pea proteins and the starch hydrolyzates.

As it will be exemplified hereinafter, the applicant company recommends using an MSD 20 tower sold by the company Niro.

The injection nozzle is chosen so as to obtain a pressure of between 50 and 300 bar, preferably about 150 bar, for a flow rate of between 100 and 1501/h, preferably about 120 l/h.

The inlet air temperatures are set in the following way:
for the inlet air upstream of the top of the tower: temperature between 150 and 180° C., preferably 155° C.,
for the static fluidized bed: temperature between 50 and 120° C., preferably 84° C.,
for the vibrated fluidized bed: temperature about 20° C.
The outlet temperature is then between 55 and 80° C., about 60° C.

The granulated powder according to the invention, containing cogranules, is finally recovered at the exit of the spray-drying tower.

According to a second variant of the invention, said granulated powder is produced according to a sole granulation process which makes it possible to carry out the step of bringing the various constituents into intimate contact. The granulation process can make use of two techniques well known to those skilled in the art: the dry granulation technique and the wet granulation technique.

According to one preferred embodiment of this second variant, the granulated powder is produced by wet granulation in a fluidized bed. An example of such a granulation is, for example, mentioned in patent EP 1 558 094, of which the applicant is the proprietor.

According to a third variant of the invention, said granulated powder is produced according to a single extrusion process. In this process, equipment comprising at least one extrusion die will be used, the temperature parameters being readily selected by those skilled in the art according to the water content of the composition before drying. The extruded composition is then successively subjected to cooling, milling and, optionally, sieving so as to result in the spray-dried powder according to the present invention.

The implementation of the drying processes described above, or processes for drying by any other drying means known to those skilled in the art, and under conditions suitable for the chosen equipment, produces a granulated powder composed of cogranules and containing the various starting compounds intimately linked to one another.

The average size of the powder produced in accordance with the invention can be characterized by its volume mean diameter (arithmetic mean) D4,3. It is between 10 μm and 500 μm, preferably between 50 μm and 350 μm, and even more preferably between 70 μm and 250 μm. According to one preferred embodiment, the volume mean diameter D4,3 of said granulated powder is between 150 μm and 240 μm.

These values are determined on an LS 230 Laser diffraction particle size analyzer from the company Beckman-Coulter, equipped with its powder dispersion module (dry process), according to the technical manual and the specifications of the constructor. The measuring range of the LS 230 Laser diffraction particle size analyzer is from 0.04 μm to 2000 μm.

According to one particular embodiment of the present invention, 90% of the powder has a diameter of less than 1000 μm, preferably less than 500 μm, and even more preferably less than 400 μm. In particular, 90% of the powder has a diameter of less than 370 μm. This value corresponds to the d₉₀.

According to another particular embodiment of the present invention, 50% of the powder has a diameter of less than 500 μm, preferably less than 300 μm, and even more preferably less than 250 μm. In particular, 50% of the powder has a diameter of less than 220 μm. This value corresponds to the d₅₀.

According to another particular embodiment of the present invention, 10% of the powder has a diameter of less than 300 μm, preferably less than 200 μm, and even more preferably less than 150 μm. In particular, 10% of the powder has a diameter of less than 100 μm. This value corresponds to the d₁₀.

These three values d₉₀, d₅₀ and d₁₀ are also determined by means of the laser diffraction particle size analyzer used for determining the volume mean diameter D4,3.

According to one preferred embodiment of the present invention, the granulated powder consists of pea protein and of starch hydrolyzates.

According to another preferred embodiment of the invention, the granulated powder contains pea proteins associated with maltodextrins, the DE of which is between 5 and 19, and preferably between 15 and 19, and glucose syrups, the DE of which does not exceed 47, and preferably 35, and/or a mixture thereof.

According to the invention, the granulated powder contains varying proportions of vegetable proteins and of starch hydrolyzates.

According to one preferred embodiment, the weight ratio of the vegetable protein, and preferably of the pea protein, to the hydrolyzate is between 90:10 and 10:90, preferably between 75:25 and 25:75, more preferably between 65:35 and 35:65. In particular, said ratio is between 55:45 and 45:55.

Thus, according to the present invention, two parameters are to be considered in the vegetable protein/starch hydrolyzate matrix. First of all, the first variable parameter is the ratio of each constituent relative to the other, and the second is the DE of the starch hydrolyzate used. Thus, for an identical ratio, several compositions of granulated powder can be obtained according to the present invention, according to the DE of the starch hydrolyzate used.

According to another preferred embodiment, the sum of the amounts of vegetable proteins, and preferably of pea proteins, and of starch hydrolyzates is between 30% and 100%, and preferably between 50% and 100%, of the total mass of said granulated powder (dry/dry).

The applicant company has, to its credit, discovered that, according to these ratios, the functional properties of the powder can be different.

In one embodiment according to the invention, it has been observed, unexpectedly, that, in the food sector, for example, the granulated powder according to the present invention has the additional advantage of completely or partially replacing the fats commonly used in recipes.

According to another embodiment of the invention, the granulated powder comprises pea proteins and starch hydrolyzates, and can also contain any suitable additive, such as flavors, dyes, stabilizers, excipients, lubricants or preservatives, provided that they do not negatively impact on the desired final functional properties.

These additives may also be pharmaceutical or phytosanitary active ingredients, or detergents. In the present invention, the term “active ingredient” is intended to mean any active molecule which has a pharmacological effect that has been demonstrated and which is of therapeutic interest, also clinically demonstrated.

Said granulated powder in accordance with the invention can also be characterized by its apparent density, determined according to the method of measurement recommended by the European Pharmacopeia (EP 5.1 volume 1, 01/2005: 20915 paragraph 2-9-15; equipment according to Figure 2-9-15-1).

Under these conditions, said granulated powder advantageously has an apparent density of between 0.30 and 0.90 g/ml, preferably between 0.40 and 0.60 g/ml.

Another functional property of the granulated powder in accordance with the invention is that it has excellent wettability, much better than the wettability noted for the simple mixture. This characteristic is the capacity for water absorption at the surface of a powder. It is proportional to the solubility of the powder and inversely proportional to the formation of lumps. A high wettability makes it possible to confer the “instant” nature on the granulated powder of the present invention.

To measure this wettability, a tall form beaker with a volume of 500 ml is used, and 250 g of distilled water at 20° C.+/−2° C. are placed in said beaker. Exactly 25 g of granulated powder in accordance with the invention or 25 g of the simple mixture are weighed out. At t=0 h, the 25 g of sample are rapidly introduced all at once, and the timer is started. The time necessary for the sample to become completely wet, i.e. for there to be no more sample in dry form, is measured. The test is carried out without stirring and with gentle stirring at 250 rpm. In the test without stirring, the granulated powder in accordance with the present invention becomes wet in less than one minute, preferably in less than 30 seconds, and even more preferably in less than 10 seconds, whereas the simple mixture takes more than 10 minutes to become completely wet.

In the test with gentle stirring, said granulated powder becomes wet in less than 30 s, preferably in less than 10 s, and even more preferably in less than 4 s, whereas the simple mixture takes more than 3 minutes to become completely wet.

In particular, and by way of example, according to the wettability test without stirring described above, a granulated powder composed of pea proteins and of maltodextrins having a DE of 19 becomes wet in less than 10 seconds, very precisely in 7 seconds, whereas the simple mixture takes 3 min 10 s to become completely wet.

This test makes it possible to demonstrate that the granulated powder has an “instant” nature compared to the simple mixture which, itself, does not have this “instant” nature.

The granulated powder of the present invention also displays a total lack of decantation, i.e. an excellent hold in suspension, which greatly facilitates its use in industrial processes, and represents a major advantage.

The hold in suspension is measured in a 250 ml graduated cylinder. After reconstitution of a 250 ml solution containing 15% of granulated powder according to the invention, in particular by resuspending said powder with gentle stirring, the volume settled out is measured every hour for 7 hours, and then after 24 h and 48 h. There is no decantation of the granulated powder, even after waiting 48 h. This total lack of decantation is not found with the simple mixture. Indeed, one hour after reconstitution of the mixture, a decantation phenomenon is observed, and accentuates with time.

Other very advantageous technological properties conferred by said granulated powder concern its emulsifying, foaming and gelling capacities, in comparison with the simple mixture of the constituents of this powder.

The emulsifying properties are due to the ability to reduce the interfacial tensions between hydrophilic and hydrophobic components of a food. They are directly related to the solubility of the protein. The powders which have these surface properties will have a considerable potential for use in emulsions in general, in refatted or nonrefatted milk powders, and also foods containing water and fats (cooked pork meats, meat, condiments).

In the present invention, the emulsifying capacity corresponds to the percentage of “emulsion cream” formed and stable after centrifugation, as a function of the amount of proteins and of the amount of oil. In order to measure it, a 50% rapeseed oil emulsion is prepared, on an Ultraturax at 9500 rpm for 1 minute, using a solution of granulated powder (hydrated for 10 minutes in demineralized water in order to be free of the ionic forces) at 2%. The emulsion is then centrifuged for 5 minutes at 1500 g. The cream volume is measured in ml. The emulsifying capacity (EC) is calculated using the following formula:

EC(as %)=(cream volume/total volume)×100

The granulated powder has an emulsifying capacity of greater than 50%, preferably greater than 55%, and even more preferably greater than 60%, whereas the simple mixtures have a low emulsifying capacity, less than 20%.

In particular, and by way of example, according to the test for measuring the EC described above, a granulated powder comprising pea proteins and maltodextrins having a DE of 19 has an EC of 87.5%.

The foaming properties, which are highly appreciated in patisseries (cakes, soufflés, meringues) and in the manufacture of mousses, based on milk or the like, and of whipped creams, are the result of partial unfolding of the proteins which orient themselves at the water/air interface.

In the present invention, the foaming capacity is measured in a 500 ml graduated cylinder. A solution containing 15% of granulated powder in accordance with the present invention is prepared on an Ultraturax at 9500 rpm for 1 minute, before being transferred into the graduated cylinder. The foam volume and the liquid volume are measured every 10 minutes for 30 minutes. The time necessary for the foam to reach 50% of its initial volume is also measured and will make it possible to quantify the stability of the foam.

The granulated powder has an excellent foaming capacity, which is extremely stable over time, whereas the simple mixture foams only very little, and gives a foam which is unstable over time.

Thus, the granulated powder has functional properties (emulsifying capacity, foaming capacity) which have been conferred thereon in particular by the process for preparing said powder.

Another very advantageous property conferred by said granulated powder according to the present invention is the clear improvement in, on the one hand, the taste and, on the other hand, the palatability and the body, which is also defined by the viscosity in the mouth.

Indeed the granulated powder has a neutral taste, unlike the simple mixture, which can have a more marked legume taste and consequently curb certain food applications. In some applications, the palatability and the body are also improved compared with the simple mixture.

These very advantageous functional properties which do not exist with a simple mixture mean that they are destined, inter alia, for very diversified and varied applications.

Another aspect of the present invention concerns the use of the granulated powder in the fields of cosmetics, detergence, agrochemistry, industrial and pharmaceutical formulations, construction materials, drilling fluids, in fermentation, in animal feed and in food applications.

Consequently, the present invention also relates to cosmetic, detergent and agrochemical compositions, industrial and pharmaceutical formulations, construction materials, drilling fluids, fermentation media, animal nutritional compositions and food applications comprising the granulated powder according to the present invention or capable of being produced according to the implementation of the process for preparing granulated powder according to the invention as described above.

In these fields, the granulated powder according to the invention can be used in compositions as a functional agent, such as emulsifying, overrun, stabilizing, thickening and/or gelling agent, in particular for totally or partially replacing animal proteins.

Consequently, the present invention also relates to an emulsifying, overrun, stabilizing, thickening and/or gelling agent which can be used for totally or partially replacing animal proteins, comprising the granulated powder according to the present invention or capable of being produced according to the implementation of the process for preparing granulated powder according to the invention as described above.

One of the particularly advantageous and valuable uses of the present invention as a total or partial replacement for animal proteins, and more particularly milk proteins, relates to the preparation of a dairy product chosen from the group consisting of fromage frais and ripened cheeses, cheese spreads, fermented milks, milk smoothies, yoghurts, specialty dairy products, and ice creams produced from milk.

According to one preferred embodiment, the powder according to the invention is used for producing ice creams with total or partial replacement of the milk proteins, by replacing them with said powder of the present invention. The advantage of this application is exemplified hereinafter in Example 4.

According to another more preferred embodiment, the powder according to the invention is used for producing cheeses with partial or total replacement of the milk proteins.

In the present invention, the term “cheese” denotes a food obtained using coagulated milk or milk products, such as cream, and then optionally draining, possibly followed by a fermentation step and, optionally, by refining (ripened cheeses). The name “cheese” is, according to decree No. 88-1206 of Dec. 30, 1988, reserved for the fermented or nonfermented, ripened or nonripened product obtained from materials of exclusively dairy origin (whole milk, partially or totally skimmed milk, cream, fat, buttermilk), used alone or as a mixture, and totally or partially coagulated before draining or after partial elimination of their water.

The milk is acidified, generally using a bacterial culture. An enzyme, rennet, or a substitute such as, for example, acetic acid, vinegar or GDL (glucono-delta-lactone) can then be added in order to cause coagulation and form the curd and the whey.

In the present invention, the term “cheese” also denotes all processed cheeses and all processed cheese spreads. These two types of cheeses are obtained by milling, mixing, melting and emulsification, under the effect of heat and emulsifying agents, of one or more varieties of cheese, with or without the addition of milk constituents and/or other food products (cream, vinegar, spices, enzymes, etc.).

Such an application is exemplified in Example 5 hereinafter by the trial concerning processed cheese spreads.

In another preferred embodiment, the powder according to the invention is used for producing yoghurts, as total or partial replacement for milk, reconstituted milk powder or milk proteins. Such an application is exemplified in Example 6 hereinafter.

Thus, the granulated powder according to the present invention or capable of being produced according to the implementation of the process for preparing granulated powder according to the invention as described above, can be used for totally or partially replacing the milk proteins in a food formulation belonging to the group defined by fromage frais and ripened cheeses, processed cheeses or processed cheese spreads, fermented milks, milk smoothies, yoghurts, specialty dairy products, and ice creams produced from milk.

Another of the particularly advantageous uses of the powder according to the present invention concerns the production of very fine oil/water emulsions, and more particularly production of coffee whiteners.

Coffee (or tea) whiteners are very fine oil/water emulsions intended to be incorporated into an instantaneous beverage of the coffee or tea type, as an alternative to certain dairy products, such as milk or alternatively cream, said diary products having a shelf-life which is too short and being too expensive. Indeed, coffee whiteners, which exist both in liquid form and in powder form, have a longer shelf-life. They therefore perform all the functions performed by milk or cream added to coffee and make it possible to whiten the coffee into which they are incorporated, thus giving a “coffee with milk” appearance. They also reduce the bitterness of coffee. Finally, they can be useful for lactose-intolerant individuals.

A conventional coffee whitener is composed:
of glucose syrup, which serves as a support;
of fat (for instance palm oil), which is responsible for the viscosity and the whitening effect of the product, due to the scattering of light on the surface of fat globules forming the emulsion;
of emulsifier (such as monoglycerides and diglycerides) which promote the “wettability” and the dispersibility of the powder in a hot liquid;
of sodium caseinates, which are proteins that contribute in particular to the whitening effect, and having emulsifying properties and improve the taste of the product, in particular by reducing the acrid nature of the tannic acids by complexation with the latter;
of stabilizing salts.

The oil/water emulsions are generally produced in the form of powders, by means of a conventional process consisting in mixing the various constituents and forming an emulsion, carrying out a homogenization step followed by an optional pasteurization step and, finally, carrying out the drying by means of a spray-drying step.

The result obtained by means of the homogenization step is an effective reduction of the particle size to a level such that it is possible to guarantee better stability of the product. Indeed, one of the most important factors in the stability of an emulsion is the diameter of the particles. The aim of the homogenization operation is precisely to reduce this diameter as much as possible and, at the same time, to also make it as uniform as possible; this then results in an improvement in the stability and an increase in the viscosity of the medium.

The emulsifying agent(s) generally used for producing these powdered oil-in-water emulsions are caseinates, in particular sodium caseinates, optionally used in the presence of monoglycerides and/or diglycerides.

However, sodium caseinates are very expensive and less and less available on the market, and producers of these oil-in-water emulsions find themselves obliged to find replacements for sodium caseinates in order to be able to continue to provide consumers with inexpensive products.

The granulated powder according to the present invention is an excellent replacement for sodium caseinates in the abovementioned application, and is capable of producing equivalent results in terms of stability, of emulsion fineness, or else of whitening capacity of these oil-in-water emulsions.

Preferably, the use of the granulated powder according to the present invention for partially or totally replacing sodium caseinates in the production of an oil/water emulsion is even more satisfactory and conclusive when the following process is applied:
a) mixing the fat, the support and the emulsifying agent(s) with vigorous stirring in order to produce an emulsion which is as fine and as stable as possible. Generally, it will be preferred to firstly mix the fat with the emulsifying agent(s), and then to add the other constituents with vigorous stirring so as to create the emulsion;
b) optionally pasteurizing the emulsion obtained in step a);
c) completing the emulsion by means of a step for homogenizing the optionally pasteurized emulsion obtained in step b); and
d) converting the optionally pasteurized, and homogenized, emulsion obtained in step c) into powder, most commonly by atomization, optionally combined with or followed by a granulation step.

Thus, the performance levels obtained in the production of the powdered oil/water emulsions using the granulated powder of the present invention are very substantially improved insofar as, in the process for producing these oil/water emulsions, the optional pasteurization step is carried out before the homogenization step, when the industrial technical equipment allows this.

That being said, entirely satisfactory results are also obtained when the “conventional” process is used, namely when the homogenization step is carried out before the optional pasteurization step.

In particular, the use of said granulated powder makes it possible to satisfy the particular and necessary performance levels required for the oil/water emulsions used as coffee whiteners, namely good dispersion in a hot and, optionally, acidic beverage such as coffee, excellent stability in said hot beverage and the same whitening capacity as a conventional coffee whitener for approximately the same amount of product incorporated into a beverage. The emulsion fineness is an important parameter which is desired for these powdered oil-in-water emulsions, in particular for the coffee whitening effect, since the finer the fat globules, the greater the surface area on which the light can scatter, and therefore the greater the whitening effect of the product. An example of use of the granulated powder according to the present invention in the preparation of a powdered oil/water emulsion for use as coffee whiteners is exemplified hereinafter.

The present invention also relates to an oil/water, and preferably coffee or tea whitening, emulsion comprising the granulated powder according to the invention or which can be produced according to the implementation of the process for preparing granulated powder according to the invention as described above.

According to another variant, the use of said granulated powder makes it possible to satisfy the particular and necessary performance levels desired for the powdered or liquid oil/water emulsions used for animal feed, and in particular for calf feed.

Thus, in particular, such emulsions, preferably powdered emulsions, are used as fatty premixes for animal feed, in particular bovine feed, and especially in the preparation of feed products for the suckling of calves. Indeed, particular performance levels are also desired for the powdered oil/water emulsions used as fatty premixes in animal feed, in particular for the suckling of calves, namely good reconstitution of the emulsion in warm water, so as to readily obtain a liquid emulsion, good stability of the emulsion, i.e. with no phase separation of the oil phase and the aqueous phase of the emulsion, good appetence and an acceptable or even pleasant taste for the animal, so as to prompt it to consume these fatty premixes.

The invention thus extends to food formulations comprising a granulated powder according to the invention or which can be obtained according to the implementation of the process for preparing granulated powder according to the invention as described above, or comprising an emulsifying, overrun, stabilizing, thickening and/or gelling agent, which can be used for totally or partially replacing animal proteins, as described above, such as:
beverages,
dairy products (including, for example, fromage frais and ripened cheeses, processed cheeses or processed cheese spreads, fermented milks, milk smoothies, yoghurts, specialty dairy products, ice creams produced from milk),
preparations intended for clinical nutrition and/or for individuals suffering from undernourishment,
preparations intended for infant nutrition,
mixtures of powders intended for diet products, or for sportspersons,
soups, sauces and cooking aids,
meat-based products, more particularly in the fine paste and brine sectors, especially in the production of hams,
fish-based products, such as surimi-based products,
all types of confectionary,
cereal products such as bread, pasta, cookies, pastries, cereals and bars,
vegetarian products and ready meals.

The granulated powder according to the present invention or which can be obtained according to the implementation of the process for preparing granulated powder according to the invention as described above also finds applications in animal feed.

The invention will be understood more clearly on reading the examples which follow, which are meant to be nonlimiting illustrations referring only to certain embodiments and certain advantageous properties according to the invention.

### Example 1

### Preparation of a Granulated Powder According To the Invention

A granulated powder containing 45% of pea proteins and 55% of maltodextrins having a DE of 19 was prepared in the following way.

The pea proteins used are sold by the applicant under the name Nutralys® S 85 M. Their total protein content is 85%.

The maltodextrins used belong to the Glucidex® range, also sold by the applicant, and are the Glucidex® maltodextrins having a DE of 19.
First of all, a suspension was prepared at a protein/maltodextrin ratio of 45/55 in a stirred tank and at a temperature of 50° C.

The mixture has a DM (dry matter content) of 35%.

The mixture obtained was homogenized on a two-stage high-pressure homogenizer (150 bar on the 1^{st} stage and 50 bar on the 2^{nd}) before being spray-dried, in order to have a perfectly homogeneous mixture.

The mixture was spray-dried in a spray-drying tower of MSD type equipped with a high-pressure spray-drying nozzle with recycling of the fine particles at the top of the tower.

### The Spray-Drying Conditions are the Following:

The injection nozzle was chosen so as to obtain a pressure of 220 bar for a flow rate of 120 l/h.
The air used was at 6 g/kg of moisture.
The air inlet temperatures were set in the following way:
   for the inlet air upstream of the top of the tower: temperature of 180° C.,
   for the static fluidized bed: temperature of 50/55° C.,
   for the vibrated fluidized bed: temperature of about 20° C.
The outlet temperature was 58° C.
The speed of the air upstream was set at 14.7 m/s, and that of the air of the static fluidized bed was 11 m/s.

The granulated powder obtained according to Example 1 exhibited the following characteristics:
Moisture content: 7%
Dry matter content: 93%
Volume mean diameter D4,3: 200 μm.

### Example 2

### Measurement of Gelling Capacity

The gelling capacity of the granulated powder obtained according to Example 1 was compared with the gelling capacity of the simple mixture of powder, using the same two constituents, and also the same ratio, as those used to prepare the granulated powder.

### 1. Solution Preparation

A solution with a concentration of 8% was prepared by placing 8 g of sample (granulated powder or simple mixture of powders) in 100 g of distilled water at 20° C.+/−1° C. 0.3 g of xanthan gum was added to the above solutions in order to avoid decantation of the particles under gravity. The mixture was stirred slowly for 30 min at a speed of 250 rpm in order to allow optimum hydration of the proteins contained in the samples.

### 2. Measuring Material

The gelatinization of the samples during a heat cycle was characterized, in the oscillatory dynamic mode, by means of the Physica® MCR301 rheometer (Anton Paar) with a striated parallel plate geometry in order to avoid sliding phenomena.

### 3. Measuring Protocol

1 ml of the hydrated suspension, prepared in paragraph (1), placed between the 50 mm diameter parallel plates, was subjected to a sinusoidal type stress, at the frequency of 1 Hertz and a deformation amplitude of 0.1 to 0.5%, while at the same time applying the following thermal cycle:
1. Heating from 20 to 90° C. in 2000 s-0.5% deformation,
2. Hold at 90° C. for 3600 s-0.2% deformation,
3. Cooling from 90 to 4° C. in 2000 s-0.1% deformation,
4. Hold at 4° C. for 12 000 s-0.1% deformation.

### 4. Interpretation

Monitoring of the storage modulus G′ and dissipation modulus G″ levels made it possible to characterize the gelling kinetics of the protein under the effect of heat and also the relative level of the force of the gel obtained.

The curves obtained made it possible to measure the gelling speed and the force of the gel obtained, but also the behavior of the gel under cold conditions.

The curves obtained with the granulated powder, in comparison with the curves obtained with the simple mixture, exhibited a faster gelling speed, a higher maximum level, which means that the gels were more solid, and also a better texture and resistance of the gel with respect to cold conditions.

This means that the gelling capacity of the granulated powder was much better than the gelling capacity of the simple physical mixture.

### Example 3

### Preparation of an Oil/Water Emulsion (Coffee Whiteners)

In this example, the granulated powder obtained according to Example 1 was used to prepare a powdered oil/water emulsion, and was compared with a control powder based on sodium caseinate.

Ingredients of the Emulsion Comprising the Granulated Powder According to the Invention (as Percentage of Ingredients Used)
61.65% of support (Roquette glucose syrup 3072)
30% of fat (palm oil)
5.55% of granulated powder obtained according to Example 1
2% of stabilizing salts (dipotassium hydrogen phosphate)
0.8% of monoglycerides and diglycerides.

Ingredients of the Control Emulsion Comprising Sodium Caseinates (as Percentage of Ingredients Used
0 64.7% of support (Roquette glucose syrup 3072)
30% of fat (palm oil)
2.5% of sodium caseinate
2% of stabilizing salts (dipotassium hydrogen phosphate)
0.8% of monoglycerides and diglycerides.

Process for Producing the Powdered Oil/Water Emulsions

a) The water and the glucose syrup were mixed and brought to 65° C. in a beaker placed in a water bath.

In parallel, the palm oil was melted in another beaker at a temperature of 65° C. The monoglycerides and diglycerides were dispersed in the oil while it was melting.

When the water/glucose syrup mixture reached the desired temperature, the powdered products (granulated powder according to the invention or sodium caseinates, stabilizing salts) were added thereto and the whole was mixed using a Kenwood® mixer, at a speed of 10 000 rpm. The molten palm oil containing the monoglycerides and diglycerides was gradually added to the water/glucose syrup/powders mixture, with stirring using a Polytron® mixer, at a speed of 4000 rpm.

The resulting composition had a water content of 50%.

b) The mixture was pasteurized at 80° C. for about 10 seconds, so as to eliminate the bacteria capable of developing in the product, but also in order to increase the stability of the emulsion that will be obtained at the end of the process.

c) This pasteurized mixture was homogenized using a Niro® Soavi (GEA group) homogenizer.

The first stage was set at a pressure of 170 bar and the second at a pressure of 30 bar.

The stability of these emulsions was observed before spray-drying. For this, the emulsion was left at ambient temperature for one hour without stirring.

If phase separation was observed, the emulsion was not stable.

The particle size (size of the fat globules) of the emulsion before spray-drying was measured using a Beckman Coulter® laser particle size analyzer coupled to a computer. This apparatus made it possible to measure the fat globule size distribution.

d) The resulting emulsion was spray-dried. The spray-drying was carried out in a spray dryer with the temperature of the air entering the spray dryer being 200° C. and the temperature of the product leaving the spray dryer being 95° C.

The resulting powdered oil/water emulsions were characterized by:
measuring the size of the fat globules of these emulsions by laser particle size analyzing as specified above.
Testing reconstitution of these emulsions in water at 80° C. or coffee at 80° C. (Nescafe® instant coffee and Carte Noire® filter coffee) and observing the stability of these reconstituted emulsions (precipitation or nonprecipitation of the proteins).

For this, the reconstituted emulsion was left at ambient temperature for one hour without stirring.
Measuring the whitening capacity of these emulsions by reconstituting these emulsions in coffee at 80° C. (Nescafe® instant coffee and Carte Noire® filter coffee) and measuring using a colorimeter.
Assessing the taste of these emulsions by means a panel of assessors using sensory analysis.

Results
measuring the size of the fat globules
   The fat globule size obtained in the two emulsions (the emulsion prepared with the granulated powder and the control emulsion) were approximately the same. The emulsions obtained were relatively fine, with an average fat globule size of approximately 2 μm.
testing reconstitution and observing stability
   It was observed that the powdered oil/water emulsion comprising the granulated powder of the present invention did not undergo phase separation in water at 80° C. and in coffee at 80° C. (Nescafe® instant coffee and Carte Noire® filter coffee), and had the same behavior as the control oil/water emulsion comprising the sodium caseinates.
Measuring the whitening capacity
   The powdered oil/water emulsion comprising the granulated powder of the present invention had a whitening capacity that was about the same as the sodium caseinate-based powdered oil/water control emulsion.
Assessing taste
   The sodium caseinate-based powdered oil/water emulsion had a taste that was judged to be very acceptable by the panel of assessors in sensory analysis.

The powdered oil/water emulsion comprising the granulated powder of the present invention had a taste which was judged to be acceptable by the panel of assessors using sensory analysis.

Conclusion

The powdered oil/water emulsion comprising the granulated powder of the present invention has characteristics similar to the sodium caseinate-based powdered oil/water emulsion. These characteristics of the emulsions are necessary for use as a coffee whitener.

Thus, the granulated powder of the present invention, comprising at least one vegetable protein and at least one starch hydrolyzate, is a good replacement for sodium caseinate in the powdered oil/water emulsions used as a coffee whitener.

### Variant of Example 3

Example 3 was reproduced in a manner identical to that described in Example 3, with the support (Roquette glucose syrup 3072) being replaced with Glucidex® 19 maltodextrins, sold by the company Roquette, and the palm oil being replaced with coconut oil. The results obtained were as advantageous as those previously obtained.

### Example 4

### Preparation of Flavored Ice Creams with Total Replacement of the Milk Proteins

In this example, the granulated powder was obtained according to the protocol carried out in Example 1, this time using a pea protein composition/maltodextrin weight ratio of 70/30.

The granulated powder therefore contains 70% of a composition of pea proteins (at a total protein content of 85%) and 30% of maltodextrins having a DE of 19.

The ice creams were prepared according to the recipes represented in the table below, and the final products were tasted, graded and compared by a sensory analysis jury.

Two ice cream recipes were tested, one being caramel ice cream the other being chocolate ice cream.

4 samples were therefore obtained:
CONTROL 1: Caramel-flavored ice cream, prepared from whole milk and flavored with a caramel flavor (Symrise, ref. 186745),
TRIAL 1: Caramel-flavored ice cream, flavored with a caramel flavor (Symrise, ref. 186745), and no longer containing milk, but containing the granulated powder according to the invention,
CONTROL 2: Chocolate-flavored ice cream prepared with whole milk and chocolate powder and flavored with a chocolate flavor (Symrise, ref. 225962),
TRIAL 2: Chocolate-flavored ice cream, flavored with a chocolate flavor (Symrise, ref. 225962), and no longer containing milk, but containing the granulated powder according to the invention.

### 1. Recipes

| | Caramel- | Caramel- | Chocolate- | Chocolate- |
|---|---|---|---|---|
| | flavored | flavored | flavored | flavored |
| | ice cream | ice cream | ice cream | ice cream |
| Ingredients (%) | CONTROL 1 | TRIAL 1 | CONTROL 2 | CONTROL 2 |
| Water | 0 | 59.75 | 0 | 59.75 |
| Whole milk | 69.75 | 0 | 69.75 | 0 |
| Chocolate | 0 | 0 | 4 | 4 |
| powder | | | | |
| Caramel flavor | 1 | 1 | 0 | 0 |
| 186745 | | | | |
| Chocolate | 0 | 0 | 1 | 1 |
| flavor 225962 | | | | |
| Sucrose | 12 | 12 | 10 | 10 |
| Glucose syrup | 8 | 8 | 6 | 6 |
| DE 40, 80% DM | | | | |
| Coconut oil | 9 | 9 | 9 | 9 |
| (vegetaline) | | | | |
| Granulated | 0 | 10 | 0 | 10 |
| powder of the | | | | |
| invention | | | | |
| Emulsifier | 0.25 | 0.25 | 0.25 | 0.25 |
| 709 VEG | | | | |
| TOTAL | 100 | 100 | 100 | 100 |

### 2. Procedure

Mix the powders dry, weigh out the emulsifier last, mixing it with the sugar.
Disperse the mixture of powders in the whole milk at 45° C. (controls 1 and 2) or in the water (tests 1 and 2) at 45° C. for 20 minutes.
Then incorporate the fats and the glucose syrup.
Leave to stir for 15 minutes.
Pasteurize at 80° C. for 3 minutes, cool, then pass the mix (temperature between 65 and 70° C.) through a homogenizer at a pressure of 250 bar.
Add the flavor and leave to mature with slow stirring at a temperature of 4° C. for a minimum of 6 hours.
Overrun to 100% and freeze in a freezer.
Deep-freeze at −30° C. for 2 hours.
Store at −20° C.

### 3. Sensory Analysis Tests

The caramel and chocolate ice cream samples were tasted blind by a jury of experts in sensory analysis, made up of 20 individuals.

The first test consisted of a triangular test in which, out of the three samples proposed, two were identical. 75% of the individuals having participated in the test were unable to recognize which were the two identical samples, this being the case for the two flavors tested.

None of the samples tested received a significant preference on the jury.

The second test, still carried out blind, consisted in tasting the various samples and in describing them. The descriptors used were identical for the ice creams containing milk proteins and for those not containing them (smoothness, fullness in the mouth, creaminess).

These two series of sensory analysis tests demonstrate perfectly that the trained jury was not able to tell the difference between a milk-based ice cream and an ice cream no longer containing milk proteins, but a granulated powder according to the present invention, or capable of being produced according to the implementation of the process for preparing granulated powder according to the invention as described above.

This invention will in particular allow individuals allergic to milk proteins to be able to taste ice creams which are as nice and creamy as their equivalents containing milk.

### Example 5

### Preparation of Processed Cheese Spread with Replacement of 10% of Milk Proteins

In this example, the granulated powder identical to that of Example 4 above was used.

This granulated powder therefore contained 70% of a composition of pea proteins (at a total protein content of 85%) and 30% of maltodextrins having a DE of 19.

The processed cheese (TRIAL) was prepared according to the recipe represented in the table below, and contains the granulated powder of said invention. It was then compared with a control cheese (CONTROL) prepared in parallel and under the same conditions and not containing the granulated powder according to the present invention.

### 1. Recipes

| | CONTROL | TRIAL |
|---|---|---|
| Cheddar (70% DM) | 32.73 | 31.70 |
| Rennet casein | 7.14 | 5.70 |
| Butter | 9.06 | 9.06 |
| Melting salt (Joha S9) | 1.20 | 1.20 |
| Melting salt (Joha S4) | 1.20 | 1.20 |
| Melting salt (Joha Tneu) | 0.13 | 0.13 |
| Granulated powder | 0 | 3.20 |
| according to the | | |
| invention | | |
| Water | 48.54 | 48.50 |
| TOTAL | 100 | 100 |
| Degree of replacement of | — | 10.4 |
| milk proteins | | |

### 2. Procedure

Preheating of the double jacket of the cooker by injection of steam (Stephan) to 100° C.
Addition of the ingredients and stirring at 300 rpm for 30 seconds.
Stirring at 3000 rpm while heating to 95° C. Holding for 3 minutes at 95° C.
Packaging in tubs and holding at ambient temperature for 24 hours.
Cooling and storage at 4° C.

### 3. Nutritional Values of the Two Cheeses

Nutritional value on 100 g of product (at use)

| | CONTROL | TRIAL |
|---|---|---|
| Total proteins | 14.4 | 14.3 |
| of which milk proteins | 14.4 | 12.9 |
| of which vegetable proteins | 0 | 1.4 |
| Carbohydrates | 0.47 | 1.14 |
| Lipids | 18.5 | 18.2 |

### 4. Other Parameters

| | CONTROL | TRIAL |
|---|---|---|
| pH | 6.1 | 6.2 |
| Theoretical dry | 40.90 | 37.60 |
| matter content | | |
| Moisture content | 74.20 | 76.40 |
| on defatted cheese | | |

The example above demonstrates perfectly that it is entirely possible to replace a part of the milk proteins with the composition of the present invention without, however, significantly modifying the nutritional values. It terms of the taste, the two cheeses were tasted by a trained jury of 20 individuals and were judged to be similar and very satisfactory.

### Example 6

### Preparation of Yoghurts Containing the Granulated Powder According to the Present Invention

In a first series of tests, trials were carried out by replacing the milk with the granulated powder or with the simple mixture of the two constituents. Two replacement percentages were tested: 10% and 50%.

In the second series of tests, the replacing of the milk in yoghurts, with a granulated powder according to the present invention, was carried out at varying replacement percentages: 10, 20, 30, 40 and 50%.

Finally, in a third and last series of trials, the replacement of the milk at a rate of 50% was tested for granulated powders according to the present invention which have varying pea/maltodextrin weight ratios. Three pea/maltodextrin weight ratios were tested: 45/55, 30/70 and 60/40.

The texture, the color and the taste were the parameters measured and compared for the three series of trials.

The procedure for preparing the various yoghurts was the same in the three series of trials, and consisted in:
Dissolving the powders in the milk preheated to 50° C.
Passing through the Niro® Soavi (GEA group) high-pressure homogenizer for a few seconds.
Carrying out a step of pasteurization of the mixture at 90° C. for 20 minutes.
Packaging in 500 ml bottles and leaving to cool to 42° C. in a water bath.
Incorporating the pre-prepared ferment: incorporate a sachet of ferment in 200 ml and leave to stir for 30 minutes, add 1 ml of ferment per 500 ml of solution.
Leaving the whole to ferment until a pH of 4.5 is obtained.
Smoothing the solution and packaging on exit.
Storing at 4° C.
Measuring the pH, the viscosity and the whiteness of each sample.

A. First Series of Tests

In this series, the granulated powder was obtained according to Example 1, using a pea protein/maltodextrin weight ratio of 45/55.

More specifically, the pea protein composition contained 85% of total pea proteins and the maltodextrins have a DE of 19.

In parallel, the simple mixture of the two constituents was prepared using the same proportions for the two constituents as those used to produce the granulated powder.

Recipes Used

| | | Granulated | | “Simple” | |
|---|---|---|---|---|---|
| | | powder | | mixture | |
| | | Degree of replacement | | | |
| | | 10% | 50% | 10% | 50% |
| | CONTROL | T10 | T50 | C10 | C50 |
| Commercially available skimmed | 86.8 | 82.17 | 45.65 | 82.17 | 45.65 |
| milk | | | | | |
| Skimmed milk powder | 4.5 | / | / | / | / |
| reconstituted at 10% | | | | | |
| Granulated powder according to | / | 4.63 | 41.15 | / | / |
| the invention in solution at 10% | | | | | |
| Granulated powder according to | / | 4.5 | 4.5 | / | / |
| the invention in powder form | | | | | |
| Simple mixture in solution | / | / | / | 4.63 | 41.15 |
| Simple mixture in powder form | / | / | / | 4.5 | 4.5 |
| Clearam ® CH2020 | 0.4 | 0.4 | 0.4 | 0.4 | 0.4 |
| Gelatin 200 Blooms-type A | 0.3 | 0.3 | 0.3 | 0.3 | 0.3 |
| (40% DM) | | | | | |
| Sucrose | 8 | 8 | 8 | 8 | 8 |
| Ferment YC380 | qs | qs | qs | qs | qs |
| Masking flavor | 0.07 | 0.07 | 0.07 | 0.07 | 0.07 |
| Total | 100 | 100 | 100 | 100 | 100 |

The ferment for yoghurt used is sold by the company CHR Hansen A/S (Denmark) under the reference CH-YC 380.

The gelatin used comes from the company Rousselot SAS, Courbevoie (France).

The Clearam® CH2020 is sold by the applicant company and is defined as a modified starch to be cooked.

Results

| | Control | T10 | T50 | C10 | C50 |
|---|---|---|---|---|---|
| pH | 4.6 | 4.3 | 4.4 | 4.4 | 4.5 |
| Texture | slightly | gelled | gelled | liquid | liquid |
| | gelled, | smooth | smooth | granular | granular |
| | smooth | | | | |
| Color | white | off-white | off-white | off-white | off-white |

This first series of tests demonstrates, firstly, that the simple physical mixing of the two powders of pea proteins and of maltodextrin does not make it possible to obtain a firm texture in a yoghurt recipe. In other words, there is no gelling and the yoghurt remains liquid with a granular texture. On the other hand, the granulated powder according to the present invention makes it possible to obtain a gelled yoghurt with a smooth texture, at the two milk replacement percentages tested: 10% and 50%.

The four yoghurts containing pea proteins have a color which is slightly more off-white than the white color of the control yoghurt, but this remains very minor.

This trial demonstrates perfectly, firstly, that it is possible to produce yoghurts by replacing up to 50% of milk with the granulated powder of the present invention, or capable of being produced according to the implementation of the process for preparing granulated powder according to the invention as described above, and, secondly, the said powder has gelling functional properties which have been conferred upon it in particular by its process of preparation, said properties not being found with the simple mixture of the constituents.

B. second series of tests

in this Series, the Granulated Powder was Identical to that used in the first series of tests (pea protein/maltodextrin (DE of 19) weight ratio of 45/55). Each time, the composition of pea proteins used to produce the granulated powder contained 85% of pea proteins.

Recipes Used

TABLE 1

| | | GRANULATED POWDER ACCORDING | | | | |
|---|---|---|---|---|---|---|
| | | TO THE INVENTION | | | | |
| | | % replacement | | | | |
| | CONTROL | 10% | 20% | 30% | 40% | 50% |
| Solution of skimmed milk | 86.8 | 82.17 | 73.04 | 63.91 | 54.78 | 45.65 |
| at 10% | | | | | | |
| Skimmed milk powder | 4.5 | / | / | / | / | / |
| Granulated powder | / | 4.63 | 13.76 | 22.89 | 32.02 | 41.15 |
| according to the invention | | | | | | |
| in solution at 10% | | | | | | |
| Granulated powder | / | 4.5 | 4.5 | 4.5 | 4.5 | 4.5 |
| according to the invention | | | | | | |
| in powder form | | | | | | |
| Clearam ® CH2020 | 0.4 | 0.4 | 0.4 | 0.4 | 0.4 | 0.4 |
| Gelatin 200 Blooms-type A | 0.3 | 0.3 | 0.3 | 0.3 | 0.3 | 0.3 |
| (40% DM) | | | | | | |
| Sucrose | 8 | 8 | 8 | 8 | 8 | 8 |
| Ferment YC380 | qs | qs | qs | qs | qs | qs |
| Total | 100 | 100 | 100 | 100 | 100 | 100 |

Results

| | | Granulated powder | | | | |
|---|---|---|---|---|---|---|
| | Control | 10% | 20% | 30% | 40% | 50% |
| pH | 4.5 | 4.4 | 4.4 | 4.3 | 4.3 | 4.3 |
| Texture | gelled | gelled | gelled | gelled | gelled | gelled |
| | smooth | smooth | smooth | smooth | smooth | smooth |
| Color | white | off- | off- | off- | off- | off- |
| | | white | white | white | white | white |

This second series of tests demonstrate that it is entirely possible to replace the milk proteins in a yoghurt with the granulated powder of the present invention, or capable of being produced according to the implementation of the process for preparing granulated powder according to the invention as described above. The degrees of replacement can range up to 50%. The five degrees of replacement give yoghurts which are entirely satisfactory and comparable to the control yoghurt in terms of the texture, which remains gelled and smooth. The color of the five trial yoghurts is very slightly off-white, but this remains very minor.

C. Third Series of Tests

In this series, the replacement of the milk in a yoghurt at a degree of 50% is tested for granulated powders according to the present invention having varying pea/maltodextrin weight ratios: 30/70, 45/55 and 60/40. For each granulated powder tested, the composition of pea proteins used contains 85% of pea proteins, and the maltodextrin was always a maltodextrin having a DE of 19.

### Recipes Used

| | | GRANULATED POWDER | | |
|---|---|---|---|---|
| | | ACCORDING TO | | |
| | | THE INVENTION | | |
| | | Pea protein/maltodextrin | | |
| | | weight ratio | | |
| | CONTROL | 30/70 | 45/55 | 60/40 |
| Solution of skimmed milk | 86.8 | 45.65 | 45.65 | 45.65 |
| at 10% | | | | |
| Skimmed milk powder | 4.5 | / | / | / |
| Granulated powder | / | 41.15 | 41.15 | 41.15 |
| according to the | | | | |
| invention in solution | | | | |
| at 10% | | | | |
| Granulated powder | / | 4.5 | 4.5 | 4.5 |
| according to the | | | | |
| invention in powder | | | | |
| form | | | | |
| Clearam ® CH2020 | 0.4 | 0.4 | 0.4 | 0.4 |
| Gelatin 200 Blooms- | 0.3 | 0.3 | 0.3 | 0.3 |
| type A (40% DM) | | | | |
| Sucrose | 8 | 8 | 8 | 8 |
| Ferment YC380 | qs | qs | qs | qs |
| Flavor + coloring | qs | qs | qs | qs |
| Total | 100 | 100 | 100 | 100 |

Results

| | | GRANULATED POWDER | | |
|---|---|---|---|---|
| | | ACCORDING TO | | |
| | | THE INVENTION | | |
| | | Pea protein/maltodextrin | | |
| | | weight ratio | | |
| | CONTROL | 30/70 | 45/55 | 60/40 |
| pH | 4.6 | 4.3 | 4.5 | 4.5 |
| Viscosity | 16 000 | 11 200 | 56 000 | 58 000 |
| (m/Pas) | | | | |
| Texture | gelled, | slightly | gelled, | gelled, |
| | smooth | gelled, | smooth | smooth |
| | | smooth | | |
| Color | white | slightly | slightly | rather |
| | | tinted | tinted | beige |

This third series of tests demonstrates that the granulated powder having a pea protein composition/maltodextrin weight ratio of 45/55 (with a content of 85% of pea proteins in the composition and a maltodextrin having a DE of 19) is the powder which makes it possible to obtain a yoghurt which is the closest to the control yoghurt. When the content of the pea protein composition in the ratio is increased (60/40), the yoghurt obtained is very creamy but the coloration is slightly beige, which may be a handicap. At a lower ratio (30/70), the texture of the yoghurt is less gelled than the control yoghurt.

### Example 7

### Preparation of Drinkable Yoghurts Containing the Granulated Powder According to the Present Invention

In this example, the granulated powder was identical to that of series 1 and 2 of Example 6 above. The pea protein/maltodextrin (DE of 19) weight ratio was therefore 45/55. Each time, the composition of pea proteins used to produce the granulated powder contained 85% of pea proteins.

Trials were carried out by replacing the milk with the granulated powder or with the simple mixture of the two constituents. Two replacement percentages were tested: 10% and 50%.

The drinkable yoghurts (TRIAL 10 and TRIAL 50) were prepared according to recipe represented in the table below, and contain the granulated powder of said invention at the two different degrees of replacement. They were then compared with the yoghurt containing only milk and also with the control drinkable yoghurts (CONTROL 10 and CONTROL 50) prepared in parallel, under the same conditions and not containing the granulated powder according to the present invention, but the simple physical mixture of the two constituents.

The various drinkable yoghurts were tasted blind by a trained jury of 20 individuals who were experts in sensory analysis. The following parameters were tested and graded on a scale of 1 to 5, 1 being the poorest grade and 5 the best: color, odor, taste, smoothness in the mouth, consistency, general grade.

1. Recipes Used

| | | Granulated | | “Simple” | |
|---|---|---|---|---|---|
| | | powder | | mixture | |
| | | Degree of replacement | | | |
| | | 10% | 50% | 10% | 50% |
| | CONTROL | T10 | T50 | C10 | C50 |
| Commercially available | 86 | 77.5 | 43 | 77.5 | 43 |
| skimmed milk | | | | | |
| Granulated powder according | / | 8.5 | 43 | 8.5 | 43 |
| to the invention in powder | | | | | |
| form | | | | | |
| SweetPearl ™ P200 | 10 | 10 | 10 | 10 | 10 |
| Nutriose ® FB06 | 4 | 4 | 4 | 4 | 4 |
| Probiotic ferment BMY-1 | qs | qs | qs | qs | qs |
| Total | 100 | 100 | 100 | 100 | 100 |

SweetPearl™ P200 is the trade name of maltitol from the applicant company: it is a carbohydrate in crystalline powder form, derived from wheat or corn starch. Nutriose® FB06 is a soluble fiber also sold by the applicant company.

The ferment used is sold by the company CHR Hansen A/S (Denmark).

2. Procedure
The Nutriose® FB06 was dissolved in the milk.
The mixture was then pasteurized at 90° C. for 10 minutes.
This pasteurized mixture was then homogenized using a Niro® Soavi (GEA group) homogenizer, at a pressure of 180 bar.
The resulting emulsion was then cooled to 43° C. and maintained at this temperature.
The prebiotic ferments were added to the cooled mixture, and the fermentation was checked by continually measuring the pH using a pH-meter.
The fermentation was stopped when the pH of the mixture reached the value of 4.5.
The SweetPearl was then added and the mixture was homogenized with an ALM2 homogenizer, sold by the company Pierre Guerin Technologies (France).
The whole was pasteurized at 90° C. for 15 seconds, in order to eliminate the risks of microbiological contamination.
The whole was cooled to 5° C. before tasting.

3. Results

| | | Granulated | | “Simple” | |
|---|---|---|---|---|---|
| | | powder | | mixture | |
| | | 10% | 50% | 10% | 50% |
| | CONTROL | T10 | T50 | C10 | C50 |
| Color | 5 | 4 | 4 | 3 | 4 |
| Taste | 5 | 5 | 4 | 3 | 2 |
| Smoothness in the mouth | 5 | 5 | 4 | 2 | 2 |
| Consistency | 5 | 5 | 5 | 2 | 1 |
| General grade | 5 | 5 | 4 | 2 | 2 |

This trial demonstrates perfectly that it is entirely possible to replace a part of the milk proteins in a drinkable yoghurt with the granulated powder of the present invention, or capable being produced according to the implementation of the process for preparing granulated powder according to the invention as described above.

The drinkable yoghurts containing said granulated powder were judged to be very satisfactory and identical to the control drinkable yoghurt containing only milk, with a very slight, but not significant, preference for the drinkable yoghurt T10 (degree of replacement of milk proteins 10%). The two drinkable yoghurts containing the simple physical mixtures of the two constituents were judged negatively and their assessment demonstrates perfectly that they are not at all similar to the control yoghurt, both in terms of the taste, in terms of their smoothness in the mouth and in terms of their consistency (judged to be too liquid). Thus, said powder has gelling functional properties which have been conferred upon it in particular by its process of preparation, said properties not being found with the simple mixture of the constituents.

1-23. (canceled)

24. A granulated powder comprising at least one vegetable protein and at least one starch hydrolyzate, wherein said granulated powder has:
a laser volume average diameter D4,3 of between 10 μm and 500 μm, and
a dry matter content, determined after stoving at 130° C. for 2 hours, of greater than 80%.

25. The granulated powder according to claim 24, wherein the weight ratio of the vegetable protein to the starch hydrolyzate is between 99:1 and 1:99.

26. The granulated powder according to claim 24, wherein the sum of the amounts of vegetable protein and of starch hydrolyzate is between 30% and 100% of the total mass of said granulated powder (dry/dry).

27. The granulated powder according to claim 24, wherein the starch hydrolyzate is selected from the group consisting of maltodextrins, glucose syrups, dextrose and mixtures thereof.

28. The granulated powder according to claim 24, wherein the vegetable protein is a protein derived from cereal plants, oleaginous plants, leguminous plants, tuberous plants, or algae and microalgae, used alone or as a mixture and chosen from the same family or from different families.

29. The granulated powder according to claim 28, wherein the vegetable protein is a leguminous plant protein, said leguminous plant being selected from the group consisting of alfalfa, clover, lupine, pea, bean, broad bean, horse bean, lentil and mixtures thereof.

30. The granulated powder according to claim 29, wherein said leguminous plant protein is pea.

31. The granulated powder according to claim 30, wherein said powder comprises at least one pea protein and one starch hydrolysate.

32. The granulated powder according to claim 30, wherein said powder comprises a pea protein and a mixture of maltodextrins and glucose syrup.

33. The granulated powder according to claim 24, wherein said granulated powder exhibits:
an apparent density of between 0.30 and 0.90 g/ml;
a wettability of less than 60 s;
a total absence of settling out; and
an emulsifying capacity of greater than 50%.

34. A process for manufacturing a granulated powder according to claim 24, comprising conjointly drying at least two constituents and bringing at least one vegetable protein into intimate contact with at least one starch hydrolyzate wherein said step of bringing into intimate contact results in a final dry matter content, determined after stoving at 130° C. for 2 hours, of greater than 80%.

35. The process according to claim 34, wherein the vegetable protein is a pea protein and the starch hydrolyzate is a maltodextrin.

36. The process according to claim 34, wherein the process comprises a step of spray-drying a suspension of at least one vegetable protein and at least one starch hydrolyzate, said spray-drying step being followed by a step of granulation of the spray-dried powder.

37. The process according to claim 34, wherein said process comprises:
a) preparing, at a temperature between 15 and 70° C. a suspension of pea proteins and of starch hydrolyzates, in which:
said pea proteins have a soluble protein content of between 20% and 99%;
said starch hydrolyzates is a maltodextrin having a DE between 15 and 19 and glucose syrups having a DE that does not exceed 47 and any mixtures thereof;
the weight ratio of the pea proteins to the starch hydrolyzates is between 99:1 and 1:99; and
the dry matter content of the suspension is between 25% and 50%;
b) carrying out an optional step of heat treatment at high temperature and for a short period of time in order to reduce the bacteriological risks of the suspension according to a);
c) carrying out an optional step of high-pressure homogenization of the suspension obtained according to a), and independently of the optional step b);
d) maintaining said suspension of pea proteins and of starch hydrolyzates at a temperature of between 15 and 80° C. or, in the event of step b) being carried out, bringing said suspension of pea proteins and of starch hydrolyzates back to a temperature of between 15 and 80° C.;
e) spray-drying said suspension in a spray-drying tower equipped with a high-pressure spray-drying nozzle after recycling of the fine particles at the top of the tower;
f) granulating in said spray-drying tower; and
g) recovering the resulting granulated powder comprising pea protein and starch hydrolyzate.

38. An oil/water emulsion comprising the granulated powder according to claim 24.

39. A cosmetic, detergent or agrochemical composition, industrial or pharmaceutical formulations, construction material, drilling fluid, fermentation medium, animal nutritional composition or food application comprising the granulated powder according to claim 24.

40. An emulsifying, overrun, stabilizing, thickening and/or gelling agent, which can be used for totally or partially replacing animal proteins, comprising the granulated powder according to claim 24.

41. A food formulation selected from the group consisting of beverages, dairy products, preparations intended for clinical nutrition and/or for individuals suffering from undernourishment, preparations intended for infant nutrition, mixtures of powders intended for diet products or for sportspersons, soups, sauces and cooking aids, meat-based products, fish-based products, all types of confectionary, cereal products, vegetarian products and ready meals, comprising an emulsifying, stabilizing, thickening and/or gelling agent, which can be used for totally or partially replacing animal proteins, comprising a granulated powder according to claim 24.

42. The food formulation according to claim 41, wherein the dairy product is selected from the group consisting of fromage frais, ripened cheeses, processed cheeses, processed cheese spreads, fermented milks, milk smoothies, yogurts, specialty dairy products and ice creams produced from milk.
